(19) United States
(12) Patent Application Publication
(10) Pub. No.: US 2003/0181354 A1
(43) Pub. Date: Sep. 25, 2003

(54) Method for central nervous system targeting through the ocular route of drug delivery

(76) Inventor: Muhammad Abdulrazik, Jerusalem (IL)

Correspondence Address: LOWE HAUPTMAN GILMAN AND BERNER, LLP, 1700 DIAGONAL ROAD, SUITE 300 /310, ALEXANDRIA, VA, 22314, US

(21) Appl. No.: 10/354,173

(22) Filed: Jan. 30, 2003

(30) Foreign Application Priority Data
Jan. 31, 2002 (IL) 147921

(51) Int. Cl. A61K 31/00

(52) U.S. Cl. 514/1

(57) ABSTRACT

A method for targeting the central nervous system, for use in the treatment and/or prevention of central nervous system disorders and/or states, comprising administering to a subject in need of treatment an effective amount of a pharmaceutical composition by the ocular route of drug delivery.

### FIELD OF THE INVENTION

The present invention relates generally to the field of targeting central nervous system (CNS) disorders. More specifically, the present invention relates to a method for treating CNS disorders by ocular route of drug delivery. The method of the present invention allows for the achievement of effective CNS target site concentrations while avoiding systemic exposure.

### BACKGROUND OF THE INVENTION

Despite major advances in neuroscience and in the understanding of the brain, the access of many potential pharmaceutical agents to the CNS is denied due to the blood-brain barrier (BBB) and to the existence of cerebrospinal fluid (CSF) flow. This presents a significant obstacle to the administration of drugs to individuals suffering from a wide range of central nervous system disorders. The BBB is formed by endothelial cells of the brain capillaries and its primary characteristic is the impermeability of the capillary wall due to the presence of complex tight junctions and low endocytic activity. The BBB serves to maintain the homeostasis of the brain so that is can function irrespective of fluctuations in the systemic concentrations of various compounds of the body. The BBB also protects the brain from toxic agents and from certain degradation compounds otherwise present in the circulatory system.

The blood brain barrier, which functions to protect the brain, also is a cause of inefficient drug delivery to the CNS. In different brain pathologies, where it is crucial to be able to provide drugs to the brain both to target the source of the disorder and to alleviate symptoms, the BBB prevents access of pharmaceutical agents to the brain. Also, significant clearance of CSF into the venous and lymphatic circulation is a limiting factor. Thus, it has traditionally been difficult to effectively treat CNS disorders due to the BBB and the CSF.

Researchers have long sought to develop ways to effectively deliver drugs to target sites in the brain. The effect of physiochemical properties, including lipophilicity, H-bonding capacity, and molecular size and shape, on brain uptake, has been studied extensively. Different strategies have been attempted in order to enable certain pharmaceutical agents, which otherwise do not cross the blood brain barrier, to penetrate the BBB, through the use of drug delivery vectors. Also, scientists have tried to modify the actual structure of the BBB and thus enable certain drugs to pass through. Mannitol, for example, is in use as an agent that modifies the osmotic balance of the BBB. However, osmotic treatment of the blood brain barrier can cause complications such as stroke, seizures, immunological side effects, and ocular toxicity.

Other delivery strategies have included intranasal administration of drugs. However, no promising results have been produced. Thus, no suitable delivery system has been achieved for allowing delivery of drugs to the brain. There is thus a need for a system for the administration of drugs to target sites in the brain that is both efficient and effective.

Surprisingly, the inventor of the present invention has found that a conventional pharmaceutical agent, when administered by ocular route of drug delivery, provides good CNS targeting. Thus, it is the primary object of the present invention to provide a method for targeting central nervous system disorders by administering to a subject in need of treatment or prevention an effective amount of a pharmaceutical agent by ocular route of drug delivery. The method of the present invention limits systemic exposure and distribution to peripheral sites of action, thus lessening unwanted side effects and the potential for toxicity. These and other objects and advantages of the present invention will become more clearly understood and appreciated from the summary of the invention and the detailed description of the invention that follows.

### SUMMARY OF THE INVENTION

The present invention relates to a method for targeting the CNS, for the treatment and/or prevention of central nervous system disorders and/or states comprising administering to a subject in need of treatment an effective amount of a pharmaceutical composition by the ocular route of drug delivery.

According to preferred embodiments of the present invention, the central nervous system disorders and/or states are selected from the group consisting of: central nervous system ischemia, central nervous system reperfusion injury, spinal ischemia, central nervous system trauma, crushed or compressed optic nerve, headache, migraine, pain, multiple sclerosis, optic neuritis, optic neuropathies, ocular glaucomatous damage, epilepsy, convulsions, neurodegenerative diseases, Parkinson's disease, Alzheimer's disease, ataxias, dystonias, movement disorders, choreas, intracranial tumors, intracranial metastasis, intracranial infections, meningitis, central nervous system states in need of cognition enhancement, memory disorders, depression, avoidant personality disorder, anxiety, panic disorder, obsessive-compulsive disorders, phobias, impulsive disorders, cognitive disorders, mood disorders, psychoses, schizophrenia, drug abuse, chemical dependencies, drugs tolerance or withdrawal, posttraumatic stress syndrome, eating disorders, obesity, premature ejaculation, hypertension, aminoglycoside antibiotics-induced hearing loss, central nervous system drug-induced disorders and states, N-methyl-D-aspartate-induced neurodegeneration, glutamate induced excitotoxic effects on nerve cells, central nervous system metabolic disorders and states, central nervous system deficiency disorders, central nervous system disorders and states amenable to neuropeptides therapy, central nervous system disorders and states amenable to neurotrophic factors therapy, central nervous system disorders and states amenable to neuroprotective therapy, central nervous system mediated ocular glaucomatous damage, autoimmune glaucoma, central nervous system disorders and states amenable to gene-therapy, surgically-induced inflammation, trauma-induced inflammation, angiogenesis-related disorder, hypoproliferative diseases, brain or spinal cord disease, disorder or injury, conditions which can lead to excessive glutamate release, conditions which can lead to neurodegeneration, stroke, impaired blood flow in neuronal tissue, septic or traumatic shock, hemorrhage shock, arthritis, arteriosclerosis, conditions which can lead to bursting of the myelin sheath around nerves, senile dementia, Huntington's disease, Lou Gehrig's disease (ALS), addictive disorders to at least one of alcohol, nicotine, and other psychoactive substance, adjustment disorder, age-associated learning and mental disorder, Anorexia nervosa, apathy, Attention-deficit disorder due to general medical conditions, Attention-deficit hyperactivity disorder, Bipolar disorder, Bulimia nervosa, Chronic fatigue syndrome, chronic or acute stress, conduct disorder, Cyclothymic disorder, dizziness, Dysthymic disorder, Fibromyalgia and other somatoform disorders, Incontinence, Inhalation disorder, Insomnia, Intoxication disorder, Obesity, Peripheral neuropathy, Premenstrual dysphoric disorder, Psychotic disorder, Seasonal affective disorder, Sexual dysfunction, Sleep disorder including narcolepsy or enuresis, Specific developmental disorder, TIC disorders including Tourette's Disease, and Withdrawal syndrome. It is thus appreciated that all CNS-related states and disorders could be treated through the ocular route of drug delivery.

Further according to preferred embodiments of the present invention, the ocular route of drug delivery is selected from the group consisting of eye-drops, suspensions, ointments, gels, hydrogels and viscosified solution systems, gel-forming systems, lotions, sprays, liposomes, emulsions, strips, therapeutic contact lenses, membrane-bound devises, collagen shields, inserts, polymeric dosing systems, rod-like inserts, iontophoresis, anterior chamber dosing, sub-conjunctival dosing or implants, sub-tenon dosing or implants, retrobulbar dosing or implants, peri-bulbar dosing or implants, trans-septal dosing or implants, choroidal dosing or implants, ciliary-body dosing or implants, sub-retinal dosing or implants, intra-vitreal dosing or implants, intraocular implantable or injected sustained release systems, encapsulated cell technology dosing systems, transscleral drug delivery systems, optic nerve related dosing systems, infusion to ocular tissue via a pump-catheter system, drug incorporation in surgical irrigating solutions and ocular dosing of gene-therapy vectors.

Still further according to preferred embodiments of the present invention, the pharmaceutical composition is a N-methyl-D-aspartate receptor antagonist. Preferably, the N-methyl-D-aspartate receptor antagonist is memantine.

Additionally according to preferred embodiments of the present invention, the pharmaceutical composition is an alpha-2 adrenoreceptor agonist. The alpha-2 adrenoreceptor agonist may comprise any acceptable salts, vehicles, and activity enhancing conjugates. Preferably, the alpha-2 adrenoreceptor agonist is brimonidine.

Moreover according to preferred embodiments of the present invention, the alpha-2 adrenoreceptor agonist is an alpha-2 adrenoreceptor subtype specific agonist.

Further according to preferred embodiments of the present invention, the alpha-2 adrenergic agonist is selected from the group consisting of imino-imidazolines, imidazolines, imidazoles, azepines, thiazines, oxazolines, guanidines, catecholamines, and derivatives thereof.

Still further according to preferred embodiments of the present invention, the pharmaceutical composition comprises a beta-blocker.

Additionally according to preferred embodiments of the present invention, the pharmaceutical composition comprises established anti-cancer therapeutics, derivatives, prodrugs, codrugs, and any combinations thereof.

Moreover according to preferred embodiments of the present invention, the pharmaceutical composition comprises established anti-Parkinsonian therapeutics, and combinations thereof.

Further according to preferred embodiments of the present invention, the pharmaceutical composition comprises recombinant adeno-associated virus, other established gene-therapy vectors, other gene delivery systems, and any combinations thereof.

Still further according to preferred embodiments of the present invention, the pharmaceutical composition comprises zinc derivatives, magnesium derivatives, vitamins, or a multi-vitamins, or any combinations thereof.

Additionally according to preferred embodiments of the present invention, the pharmaceutical composition comprises established ophthalmic therapeutics and their combinations, derivatives, pro-drugs, and co-drugs.

Moreover according to preferred embodiments of the present invention, the pharmaceutical composition comprises one or more prostaglandine analogues, prostaglandine derivatives, pro-drugs, co-drugs, and combinations thereof. Preferably, the prostaglandine analogue is selected from the group consisting of latanoprost, unoprostone, travaprost and bimatoprost.

Further according to preferred embodiments of the present invention, the pharmaceutical composition comprises one or more prostamid receptor agonists. Preferably, the prostamid receptor agonist comprises bimatoprost.

Still further according to preferred embodiments of the present invention, the pharmaceutical composition comprises one or more one of the agonists of the cannabinoid receptors.

Additionally according to preferred embodiments of the present invention, the pharmaceutical composition comprises a steroid. Preferably, the steroid is an angiostatic steroid. More preferably, the angiostatic steroid comprises Anecortave.

Moreover according to preferred embodiments of the present invention, the pharmaceutical composition comprises an imino-imidazoline selected from the group consisting of clonidine and apraclonidine.

Further according to preferred embodiments of the present invention, the pharmaceutical composition comprises an imidazoline selected from the group consisting of naphazoline, xymetazoline, tetrahydrozoline, and tramazoline.

Still further according to preferred embodiments of the present invention, the pharmaceutical composition comprises an imidazole selected from the group consisting of detomidine, medetomidine, and dexmedetomidine.

Additionally according to preferred embodiments of the present invention, the pharmaceutical composition comprises an azepine selected from the group consisting of B-HT 920 (6-allyl-2-amino-5,6,7,8 tetrahydro-4H-thiazolo[4,5-d]-azepine) and B-HT 933.

Moreover according to preferred embodiments of the present invention, the pharmaceutical composition comprises a thiazine. Preferably, the thiazine comprises xylazine.

Further according to preferred embodiments of the present invention, the pharmaceutical composition comprises an oxazoline. Preferably, the oxazoline is rilmenidine.

Still further according to preferred embodiments of the present invention, the pharmaceutical composition comprises a guanidine selected from the group consisting of guanabenz and guanfacine.

Additionally according to preferred embodiments of the present invention, the pharmaceutical composition comprises a catecholamine.

Moreover according to preferred embodiments of the present invention, the pharmaceutical composition comprises an alpha-2 adrenergic agonist comprising at least one quinoxaline component. Preferably, the quinoxaline components comprises quinoxaline derivatives selected from the group consisting of (2-imidozolin-2-ylamino) quinoxaline, 5-halide-6-(2-imidozolin-2-ylamino) quinoxaline, and tartrates of 5-bromo-6-(2-imidozolin-2-ylamino) quinoxaline. The halide of the 5-halide-6-(2-imidozolin-2-ylamino) quinoxaline may be a fluorine, a chlorine, an iodine, or preferably, a bromine, to form 5-bromo-6-(2-imidozolin-2-ylamino) quinoxaline. Even more preferably, the derivatives of quinoxaline includes a tartrate of 5-bromo-6-(2-imidozolin-2-ylamino) quinoxaline, or brimonidine tartrate.

Further according to preferred embodiments of the present invention, the subject is an animal.

Still further according to preferred embodiments of the present invention, the subject is a human.

The present invention also relates to a method for treating migraines in humans, comprising administering to a subject in need of treatment an effective amount of a pharmaceutical composition by the ocular route of drug delivery. In some preferred embodiments, the method also comprises administering to the subject an effective amount of an established anti-migraine therapeutic agent in combination with said pharmaceutical composition. Preferably, the anti-migraine therapeutic agent is delivered through a systemic route. Alternatively, the anti-migraine therapeutic agent is delivered through the ocular route of drug delivery.

Preferably, the pharmaceutical composition comprises an alpha-2 adrenoreceptor agonist. The alpha-2 adrenoreceptor agonist may comprise any acceptable salts, vehicles, and activity enhancing conjugates. More preferably, the alpha-2 adrenoreceptor agonist comprises brimonidine.

Preferably, the alpha-2 adrenoreceptor agonist is an alpha-2 adrenoreceptor subtype specific agonist. More preferably, the alpha-2 adrenergic agonist is selected from the group consisting of imino-imidazolines, imidazolines, imidazoles, azepines, thiazines, oxazolines, guanidines, catecholamines, and derivatives thereof.

Further according to preferred embodiments of the present invention, the pharmaceutical composition comprises brimonidine tartrate. Preferably, the brimonidine tartrate is administered through the ocular route of drug delivery in a 0.0001%-9% of w/v composition.

The present invention also relates to a method for treating migraines in humans, comprising administering to a subject in need of treatment an effective amount of an established anti-migraine therapeutic agent by the ocular route of drug delivery.

It is thus appreciated that the method of the present invention will have far-reaching consequences in the field of medical treatment. It will enable CNS drug delivery to ultimately be carried out in a faster, more direct, and more effective way than was previously possible.

### BRIEF DESCRIPTION OF THE DRAWINGS

FIGS. 1-8, and Tables 1-2, illustrate the results of a neuro-ocular tissue distribution study of brimonidine tartrate after ocular dosing. While the studies provided relate to targeting of the CNS using brimonidine, it is appreciated that other pharmaceutical agents could be delivered to the CNS also through the ocular route of drug delivery, and thus the invention is not limited to brimonidine. Rather, the scope of the invention is as defined in the appended claims.

### DETAILED DESCRIPTION OF THE INVENTION

The use of ocular dosing to target CNS disorders offers several benefits over the use of systemic, or intranasal delivery strategies, because it can achieve significant CNS target site concentrations while limiting systemic exposure and distribution to peripheral sites of action, which will lessen unwanted effects and toxicity. Surprisingly, it was found that brimonidine, a conventional ophthalmic therapeutic agent, can be administered efficiently to target sites in the brain without resulting in systemic exposure. This is accomplished through the use of the ocular route of drug delivery.

The brain and the eye are both isolated and highly protected organs, sharing the sensory retina, which is considered to be a part of both organs. Whereas the blood-brain and blood-ocular barriers are well defined, the paucity of knowledge prevents the drawing of a clear ocular-brain barrier.

To date, the risks of central nervous system side effects of locally administered ophthalmic therapeutic agents are thought to be the consequence of systemic absorption of these drugs.

The conventional method of penetration of the topically instilled drugs into the various ocular tissues has always been assumed to be through the corneal route. Traditionally, little attention has been given to the non-corneal routes, like the conjunctival/scleral route, as the precise mechanism for the non-corneal penetration is not clear. Nevertheless, the penetration of drug across the conjunctiva/sclera can be significant for poorly cornea-permeable drugs. Topically applied drugs should penetrate through either the vitreous or the choroid to reach adequate levels in the retina. However, most of the drugs do not enter the vitreous in adequate amounts after ocular topical application. Following conjunctival/scleral penetration, drugs available in the choroid encounter the barrier of the junctional complexes between retinal pigment epithelium cells and the pigment epithelium cells of the pars plana, and must cross the epithelial cell membrane, usually by passive diffusion, before penetrating the retina.

The retino-geniculate pathways were studied intensively mainly through the axonal delivery patterns of radiolabled probes.

Prior art research, such as the studies about to be described, could not provide a solid and convincing case for the usage of ocular dosing as a means for administering a drug to CNS target sites. However, with the unexpected results obtained by the inventor of the present invention, it can be concluded that the ocular route of drug delivery could well be used as an efficient and effective method of drug delivery to the brain.

In one study, recombinant adeno-associated virus (rAAV) was applied to the eye in order to characterize the delivery of a transgenic protein to the CNS. High levels of green fluorescent protein (GFP, a reporter gene) persists at least 6 months in optic nerves and brains of mice and dogs after intravitreal delivery of rAAV-GFP. However, the results of this study could not lead one to the conclusion that ocular dosing is an efficient means for drug delivery to the brain, since the GFP was not detected opthalmoscopically in AAV-GFP injected eyes until 2-3 weeks after injection. The peak was at six weeks, and GFP was not detected beyond the first synapse of the ganglion cells axons in the CNS (Dudus L, Anand V, Acland G M, Chen S J, Wilson J M, Fisher K J, Maguire A M, Bennett J. Persistent transgene product in retina, optic nerve and brain after intraocular injection of rAAV.Vision Res July 1999; 39(15):2545-53).

In another study, the spread of herpes simplex virus (HSV) in the CNS after ocular inoculation was studied by autoradiographic localization of neuronal uptake of tritated thymidine. After ocular inoculation, the spread of herpes simplex virus was shown to be restricted to a small number of noncontiguous, but synaptically related foci, in the brain stem and cortex, which become infected in a sequential fashion. The authors thus presumed that the principal route of the spread of HSV in the CNS after ocular infection appears to be via axonal transport rather than by local, diffuse spread (Margolis T P, LaVail J H, Setzer P Y, Dawson C R. Selective spread of herpes simplex virus in the central nervous system after ocular inoculation. J Virol November 1989; 63(11):4756-61). These results are thus unrelated from those in the present invention, since the spread of brimonidine in the CNS points to non-axonal delivery based on the quick at speed which the drug reaches different part of the brain, whereas HSV seemingly spreads via an axonal route.

In another study, it was demonstrated that microvessels in the prelaminar region (PLR) of the optic nerve head lack classical blood-brain barrier characteristics and display nonspecific permeability, possibly mediated by vesicular transport (Hofman P, Hoyng P, vanderWerf F, Vrensen G F, Schlingemann R O. Lack of Blood-Brain Barrier Properties in Microvessels of the Prelaminar Optic Nerve Head. Invest Ophthalmol Vis Sci April 2001; 42(5):895-901). This was carried out using immunohistological staining of different regions of the optic nerve head, retro-laminar optic nerve, and retina of humans and monkeys, using antibodies against BBB markers, the non-BBB marker PAL-E, and against plasma proteins fibrinogen and IgG, which serve as endogenous markers of nonspecific microvascular permeability. It is appreciated that the findings were limited only to the PLR and did not include other investigated regions.

These and other studies have revealed only some of the characteristics of the pharmacokinetics after ocular dosing.

The following examples are presented to illustrate various aspects of the present invention, but are not intended to limit the scope of the invention in any respect, as set out in the claims.

### EXAMPLE 1

A 52-year old male patient with a history of migraine, and both eyes primary open angle glaucoma. Following the prescription of brimonidine tartrate 0.2% as a third topical antiglaucoma agent for the left eye, the patient has reported a substantial relief of migraine related symptoms.

### EXAMPLE 2

A 48-year old female patient with a history of migraine, and right eye primary open angle glaucoma. Following the prescription of brimonidine tartrate 0.2% as a second topical antiglaucoma agent, the patient has reported a substantial relief of migraine related symptoms.

### EXAMPLE 3

A study was conducted to examine for the first time the neuro-ocular tissue distribution of brimonidine following one single 50 μl instillation of 3H-Alphagan aqueous solution (0.2%) into the albino rabbit eye. Both eyes and the brain were dissected. Both side specimens of aqueous humor, cornea, iris, lens, vitreous, conjunctiva, sclera, ciliary body, choroid, retina, optic nerve, optic tract, olfactory bulb, as well as corpus callosum and blood samples were collected. The corpus callosum was chosen as an indicator of general availability of the drug in the brain. The olfactory bulbs were included to rule out ocular-brain drug delivery through the nasal cavity.

In the method employed, following single instillation of 50 μl of [3H]-radio labeled brimonidine tartrate in the cul-de-sac of the right eye, male albino rabbits (2-2.5 kg) were sacrificed at the selected time point (15, 30 min., 1, 2. 3, and 4 h). Both eyes and the brain were dissected. Both sides specimens of aqueous humor, cornea, iris, lens, vitreous, conjunctiva, sclera, ciliary body, choroid, retina, optic nerve, optic tract, as well as chiasm, cerebral and blood samples were weighted before combustion in Packard sample Oxidizer (model 307), and radioactive [3H] brimonidine liquid scintillation counting.

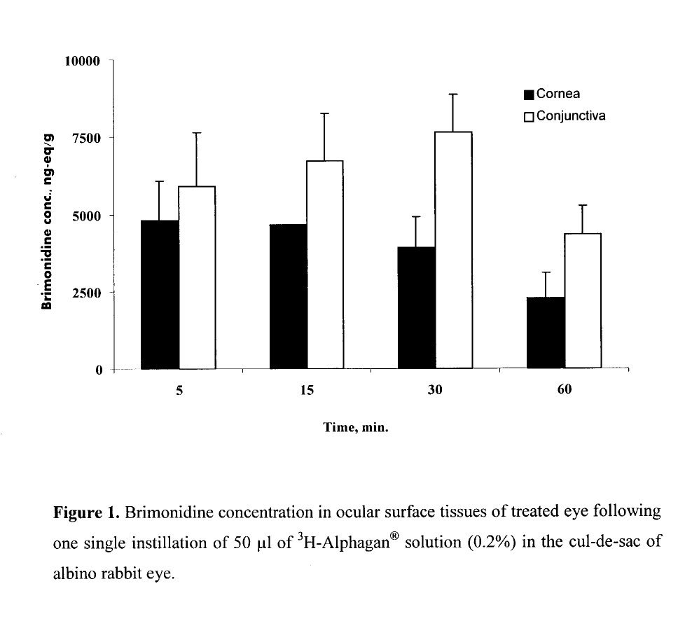

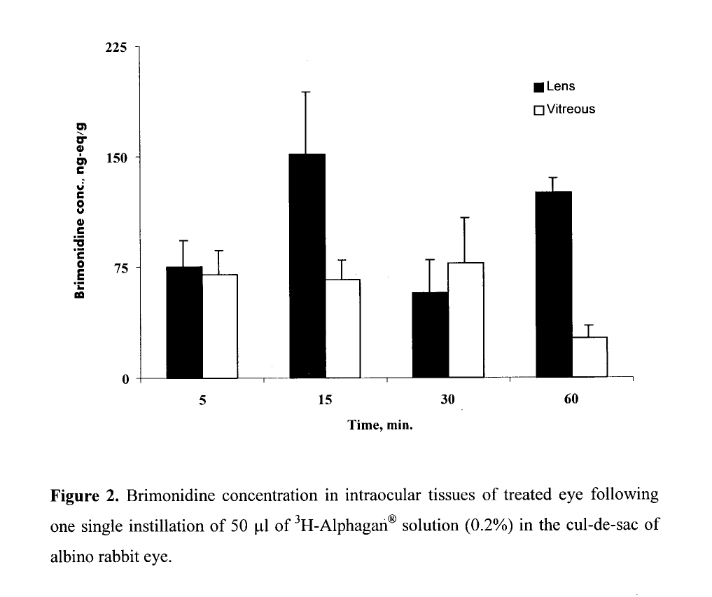

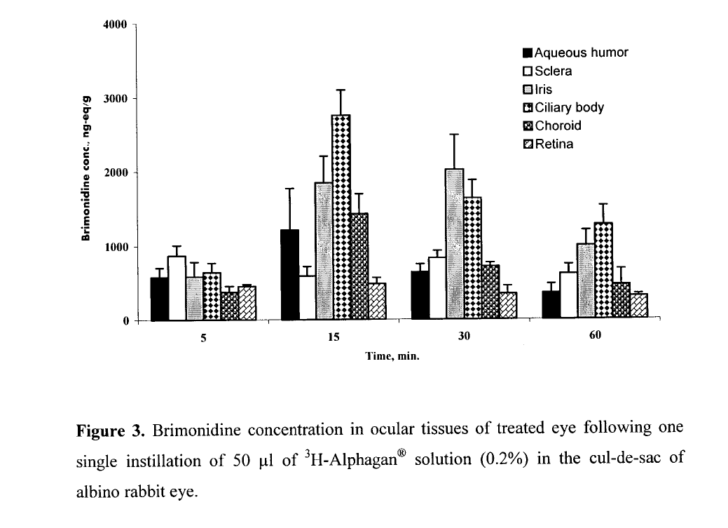

The current studies have demonstrated that topically applied brimonidine widely distributes into the anterior and posterior segments of albino rabbit eye following one single 50 μl instillation of 3H-radiolabeled Alphagan® 0.2% aqueous solution. In the treated eye the highest brimonidine levels were detected in the cornea and conjunctiva (FIG. 1), and the lowest levels in the lens and vitreous (FIG. 2). Vitreal area under curve (AUC) was only 8.85% of aqueous humor AUC (P<0.01) and 15.7% of retinal AUC (p<0.001), suggesting poor penetration to the vitreous from both the aqueous humor and the retina (Table 1). Choroidal brimonidine AUC was 196% higher than retinal AUC (p<0.001), which in turn was 636% higher than vitreous AUC (p<0.001). This clear drug level gradient, suggest that the route of brimonidine delivery to the retina following topical application is mainly through the choroid rather than the vitreous.

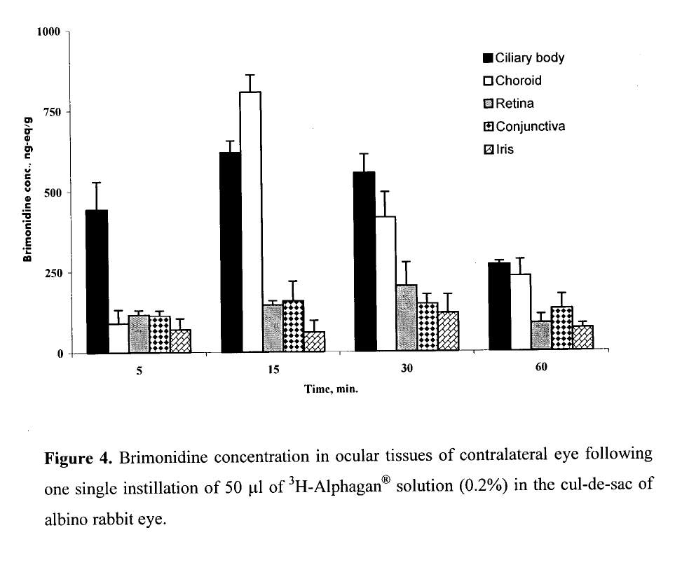

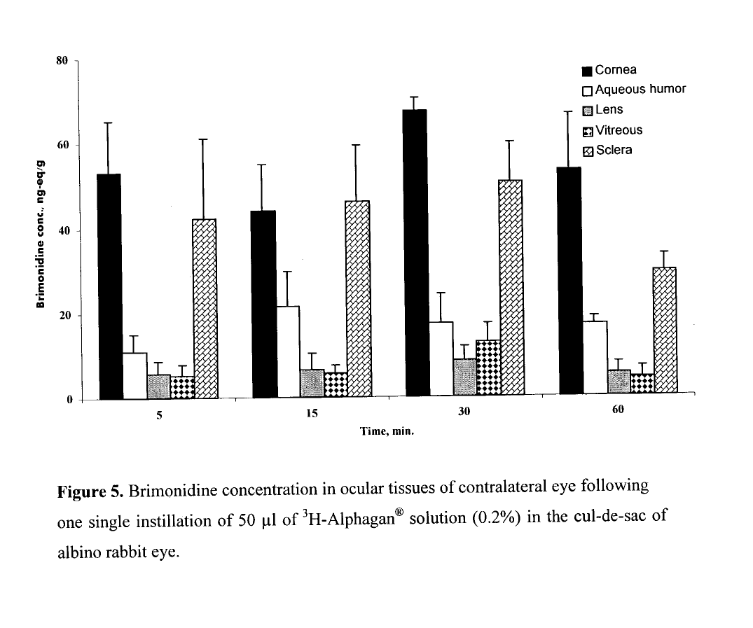

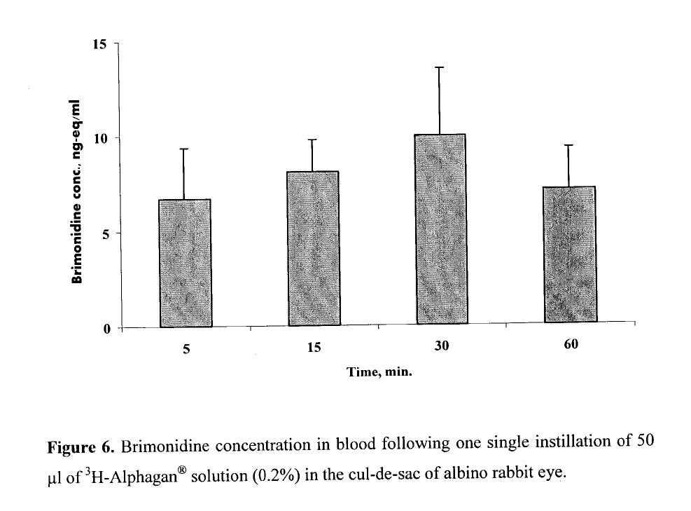

The highest brimonidine level in the contralateral eye (FIG. 4) was found in ciliary body and choroid followed by retina, conjunctiva, and iris, with very low brimonidine levels in other tissues (FIG. 5). The ciliary body brimonidine level was already relatively high (444.28±86.38 ng/g) at 5 minutes post drug application, showed a peak (619.74±36.35 ng/g) at 15 minutes, and declined moderately thereafter (FIG. 4). With the extremely low corresponding blood brimonidine levels (FIG. 6), this pharmacokinetic profile in the contralateral eye suggest regional rather than systemic route of drug delivery between the treated and the contralateral eye. Compared to other tissues of the contralateral eye (Table 1), the significantly higher brimonidine AUC in the ciliary body (322% higher than in the retina, P<0.001, and 340% higher than in the conjunctiva, P<0.001) and the choroid (275% higher than in the retina, P<0.01, and 290% higher than in the conjunctiva, P<0.01), suggests that brimonidine delivery to the contralateral eye is done mainly through its uveal tract tissues. The rapid (<5 minutes) brimonidine delivery to the ciliary body of the contralateral eye (FIG. 4) is a hint that the proposed regional drug delivery route between both eyes is most probably a vascular one that comprise a link between both eyes uveal tract tissues.

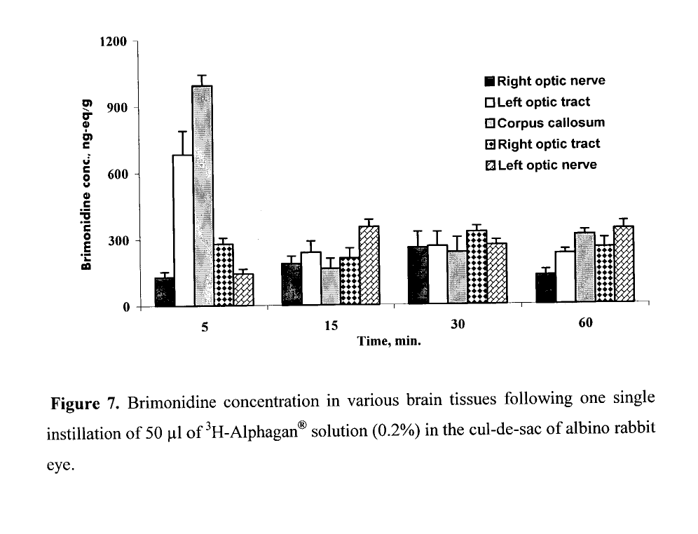

In the present study, we observed significant drug retention, after single topical application to the right eye of albino rabbit, in the right and left optic nerves and tracts as well as the corpus callosum (FIG. 7).

The extremely low drug concentration detected in the blood samples (FIG. 6) suggests that drug delivery from the treated eye to the brain was not through the systemic circulation. At 5 minutes brimonidine concentration was 148 folds higher in the corpus callosum than in the blood (P<0.001), and for the whole study period the brimonidine AUC was 40 folds higher for the corpus callosum than the blood (P<0.001). Although an early (<5 min.) brimonidine blood peak cannot be ruled out, the later (15 min.) contralateral eye peak (FIG. 4) in choroid (99.5 folds higher than in the blood, P<0.001) and ciliary body (76.5 folds higher than in the blood, P<0.001) definitely cannot be related to drug absorption from the systemic circulation. This fact, combined with the reported systemic half life of brimonidine (3 hours), makes the possibility of ocular-CNS drug delivery through the systemic circulation by a significant early (<5 min.) brimonidine peak in the blood, very unlikely.

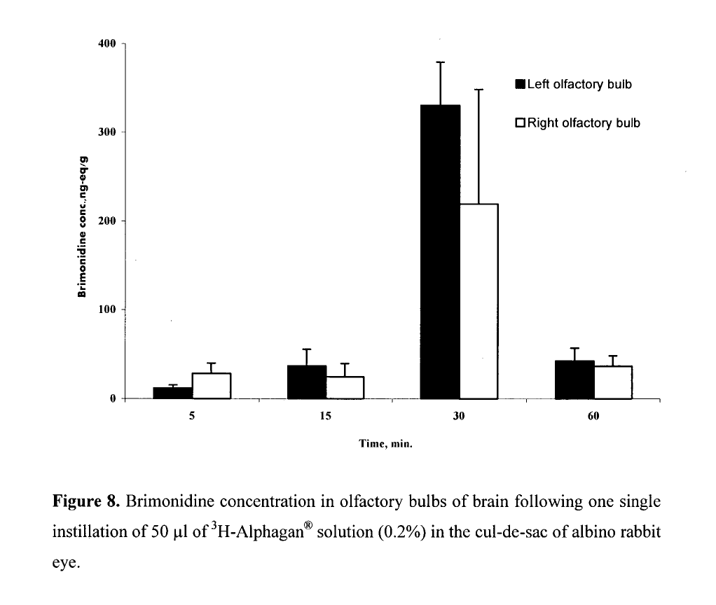

Alternatives to the systemic route of drug delivery to the brain after ocular topical application include ocular-brain axis route, comprised by the optic nerve structures, and an indirect route by drainage to the nasal mucosa and absorption by the olfactory bulbs. With the exception of fair levels at 30 min time point, drug levels in the olfactory bulbs showed non-significant variations in the low range (FIG. 8). The low drug levels observed in the olfactory bulbs contradicts the possibility of nasal-brain delivery as a major non-systemic route of drug delivery to the brain after ocular topical application. Although early brimonidine peak in the olfactory bulbs cannot be ruled out, the lowest drug levels observed in this tissue at 5 min. post dosing excludes this possibility. Moreover, since drug absorption through the nasal mucosa with its rich vasculature will lead also to systemic drug absorption, the absence of early (<5 minutes) brimonidine peak fingerprint in the blood samples makes the possibility of early brimonidine peak in the olfactory bulbs more unlikely to occur.

As proposed earlier in the discussion, the route of brimonidine delivery to the retina following topical application is mainly through the choroid. The penetration from the choroid to the optic nerve can be achieved by either drug penetration through the pigment epithelium to the retina and axoplasmic flow from retinal ganglion cell bodies, or through possible connections between the choroid and the optic nerve head structures. Investigators in a recent study have demonstrated that microvessels in the prelaminar region of the optic nerve head lack classical blood-brain barrier characteristics and display nonspecific permeability, possibly mediated by vesicular transport, suggesting possible route of drug delivery through the optic nerve head. Such drug delivery can proceed along the optic nerve through vascular structures like the pia mater, or by cerebro spinal fluid (CSF) delivery. Although CSF samples were not collected in the present study, and the possibility of CSF delivery cannot be ruled out, the mechanism of possible CSF drug delivery along the optic nerve is not clear. The centrally secreted and peripherally absorbed CSF cannot allow active bidirectional drug delivery, from the treated optic nerve head to the brain and from the brain to the contralateral optic nerve head.

At 5 min., corpus callosum 3H-brimonidine concentration was 993.79±48.43 ng/g, 763% higher than in the right optic nerve (P<0.001), suggesting fast ocular-brain drug delivery following topical ocular application. This fast brimonidine delivery to the CNS following topical application, should rule out the possibility of axonal delivery. Reportedly, fast axonal transport is in the range of few millimeters per hour. Moreover, the highest brimonidine AUC in the neuronal tissues was found in the corpus callosum (Table 2), 177% higher than in the right optic nerve (P<0.01), 118% higher than in the left optic nerve (P<0.01), 123% higher than in the right optic tract (P<0.05), and 111% higher than in the left optic tract (P<0.10), despite the fact that there are no direct connections between the optic tracts and the corpus callosum.

In conclusion, our data provide the first case of good CNS availability after ocular application of conventional ophthalmic therapeutic agent, through non-systemic routes.

It is highly likely that the pathway of drug delivery through the CNS is different depending on the particular type of compound that is being administered.

Thus, while further experimentation is needed, the results indicate that ocular dosing of conventional ophthalmic therapeutics may be useful to achieve good drug availability in the CNS. It will be understood by those of ordinary skill in the art that the same can be performed with a wide range of therapeutic agents and conditions without affecting the scope of the invention or any embodiment thereof. Accordingly, the exclusive rights sought to be patented are as described in the appended claims.

CNS Significance of Brimonidine and Alpha-2 Adrenoreceptors:

Brimonidine acts as a potent alpha 2-adrenoreceptor agonist (A2-R). It is known to effect the eye by reducing intraocular pressure (IOP) via decreasing aqueous production and increasing uveosceral flow. It also has an affinity for non-adrenergic imidazoline receptor and it may also cause a decrease in IOP (and also a decease in blood pressure) via binding to these receptors. Brimonidine is known to pass through the blood-brain barrier (BBB) and thus has the potential for CNS toxicity or CNS-mediated activity. It has been shown to have potentially dangerous side effects when administered topically to children eyes for medical therapy of glaucoma.

The following detailed discussion of research studies relating to brimonidine is meant to underscore brimonidine's influence in the central nervous system and thus its potential for usage in the treatment of various CNS states and disorders. It is appreciated, however, that the method of the present invention is not limited only to brimonidine, at that ocular dosing could be used as a suitable CNS delivery system for many other therapeutic drugs as well.

Much research has been conducted concerning the functioning of the A2-R receptors in the brain and it is clear from the available research that A2-R and brimonidine binding play a role in many neurological disorders. Degeneration of A2-R receptors has been shown to be age-related, and is characteristic of Alzheimer's patients. The ability of an A2-R agonist to improve cognitive function has been reported. Very small doses of brimonidine produced a reliable but modest improvement of memory in monkeys. A2-R may also be associated with depression, as increased A2-R agonist binding sites in have been found in the hippocampus and frontal cortex of suicidal individuals. The binding capacity of brimonidine was found to be in the frontal cortex (Bmax 30% greater), and to a lesser extent in the hypothalamus in the brain of suicide. In Alzheimer's patients, the binding capacity of brimonidine is lesser in certain areas of the brain. (Meana J J, Barturen F, Garro M A, Garcia-Sevilla J A, Fontan A, Zarranz J J. Decreased density of presynaptic alpha 2-adrenoceptors in postmortem brains of patients with Alzheimer's disease. J Neurochem May 1992; 58(5):1896-904). For the treatment of Parkinson's disease, there may be a potential benefit of A2-R antagonists. (Chopin P, Colpaert F C, Marien M. Effects of alpha-2 adrenoceptor agonists and antagonists on circling behavior in rats with unilateral 6-hydroxydopamine lesions of the nigrostriatal pathway. J Pharmacol Exp Ther February 1999; 288(2):798-804).

Yet other research has disclosed the effect of brimonidine on compounds in the brain, including oxytocin, acetylcholine, serotonin, and norepinephrine. (Diaz-Cabiale Z, Narvaez J A, Petersson M, Uvnas-Moberg K, Fuxe K. Oxytocin/alpha(2)-Adrenoceptor interactions in feeding responses. Neuroendocrinology March 2000; 71(3):209-18), (Diaz-Cabiale Z, Petersson M, Narvaez J A, Uvnas-Moberg K, Fuxe K. Systemic oxytocin treatment modulates alpha 2-adrenoceptors in telencephalic and diencephalic regions of the rat.Brain Res Dec. 29, 2000; 887(2):421-5). It has been shown that A2-R, both on noradrenergic neurons (autoreceptors) and on non-noradrenergic cells (heteroreceptors), can participate in mediating drug-induced changes in medial prefrontal cortical acetylcholine release (Tellez S, Colpaert F, Marien M. Alpha2-adrenoceptor modulation of cortical acetylcholine release in vivo. Neuroscience 1999;89(4):1041-50). Numazawa et al, reported the inhibitory action of brimonidine on the release of serotonin (5-HT) from the rat hippocampus in vivo (Numazawa R, Yoshioka M, Matsumoto M, Togashi H, Kemmotsu O, Saito H Pharmacological characterization of alpha 2-adrenoceptor regulated serotonin release in the rat hippocampus. Neurosci Lett Jun. 16, 1995; 192(3):161-4). In another study, the results suggested that alpha 2B-subtype receptors mediate norepinephrine hyperalgesia while the antinociceptive effect of alpha 2-agonist is mediated by the alpha 2C-subtype receptor (Khasar S G, Green P G, Chou B, Levine J D. Peripheral nociceptive effects of alpha 2-adrenergic receptor agonists in the rat. Neuroscience May 1995; 66(2):427-32).

TABLE 1
Summary of ocular pharmacokinetic data after a one single 50 μ1 instillation of
³H-radiolabeled Alphagan ® solution (0.2%) into the right eye of albino rabbits
(N = 3 eyes at each of four post-dosing time points, ± SD)

| | Treated eye | | | Contralateral eye | | |
|---|---|---|---|---|---|---|
| | Tₘₐₓ | Cₘₐₓ | AUC_{0-60 min} | Tₘₐₓ | Cₘₐₓ | AUC_{0-60 min} |
| Tissues/Fluids | min | ng/g | ng.min.g⁻¹ | min | ng/g | ng.min.g⁻¹ |
| Conjunctiva | 5 | 7331.87 ± | 249063.97 ± | 15 | 170.32 ± | 8148.95 ± |
| | | 3060.43 | 182337.44 | | 57.58 | 3186.78 |
| Ciliary body | 15 | 2746.09 ± | 95077.29 ± | 15 | 620.33 ± | 27693.64 ± |
| | | 294.90 | 2391.34 | | 30.74 | 1665.97 |
| Choroid | 15 | 1426.33 ± | 43617.30 ± | 15 | 805.55 ± | 23606.98 ± |
| | | 216.36 | 1754.17 | | 53.53 | 3524.76 |
| Iris | 30 | 2264.92 ± | 87779.50 ± | 30 | 122.03 ± | 5354.37 ± |
| | | 156.10 | 6191.60 | | 57.10 | 2088.23 |
| Retina | 15 | 510.87 ± | 22170.73 ± | 30 | 209.28 ± | 8585.92 ± |
| | | 58.47 | 2207.51 | | 50.59 | 1819.00 |
| Vitreous | 5 | 88.76 ± | 3488.16 ± | 30 | 12.94 ± | 465.48 ± |
| | | 14.07 | 849.55 | | 5.43 | 163.51 |

TABLE 2
Summary of pharmacokinetic data of optic axonal tissues in brain
after a one single instillation of ³H-radiolabeled Alphagan ®
solution (0.2%) into the right eye of albino rabbits (N = 3, ± SD)

| | Tₘₐₓ | Cₘₐₓ | AUC_{0-60 min} |
|---|---|---|---|
| Tissues | min | ng/g | ng.min.g⁻¹ |
| Right optic nerve | 30 | 272.64 ± 47.31 | 11049.54 ± 1050.20 |
| Left optic tract | 5 | 682.39 ± 108.56 | 17595.28 ± 586.80 |
| Corpus callosum | 5 | 993.79 ± 48.43 | 19537.11 ± 1363.09 |
| Right optic tract | 30 | 327.60 ± 23.36 | 15932.49 ± 1454.38 |
| Left optic nerve | 15 | 355.39 ± 25.91 | 16621.08 ± 476.53 |

1. A method for targeting the central nervous system, for use in the treatment and/or prevention of central nervous system disorders and/or states, comprising administering to a subject in need of treatment an effective amount of a pharmaceutical composition by the ocular route of drug delivery.

2. A method according to claim 1, wherein the central nervous system disorders and/or states are selected from the group consisting of: central nervous system ischemia, central nervous system reperfusion injury, spinal ischemia, central nervous system trauma, crushed or compressed optic nerve, headache, migraine, pain, multiple sclerosis, optic neuritis, optic neuropathies, ocular glaucomatous damage, epilepsy, convulsions, neurodegenerative diseases, Parkinson's disease, Alzheimer's disease, ataxias, dystonias, movement disorders, choreas, intracranial tumors, intracranial metastasis, intracranial infections, meningitis, central nervous system states in need of cognition enhancement, memory disorders, depression, avoidant personality disorder, anxiety, panic disorder, obsessive-compulsive disorders, phobias, impulsive disorders, cognitive disorders, mood disorders, psychoses, schizophrenia, drug abuse, chemical dependencies, drugs tolerance or withdrawal, posttraumatic stress syndrome, eating disorders, obesity, premature ejaculation, hypertension, aminoglycoside antibiotics-induced hearing loss, central nervous system drug-induced disorders and states, N-methyl-D-aspartate-induced neurodegeneration, glutamate induced excitotoxic effects on nerve cells, central nervous system metabolic disorders and states, central nervous system deficiency disorders, central nervous system disorders and states amenable to neuropeptides therapy, central nervous system disorders and states amenable to neurotrophic factors therapy, central nervous system disorders and states amenable to neuroprotective therapy, central nervous system mediated ocular glaucomatous damage, autoimmune glaucoma, central nervous system disorders and states amenable to gene-therapy, surgically-induced inflammation, trauma-induced inflammation, angiogenesis-related disorder, hypoproliferative diseases, brain or spinal cord disease, disorder or injury, cnditions which can lead to excessive glutamate release, cnditions which can lead to neurodegeneration, stroke, iaired blood flow in neuronal tissue, sptic or traumatic shock, hemorrhage shock, arthritis, arteriosclerosis, conditions which can lead to bursting of the myelin sheath around nerves, senile dementia, Huntington's disease, Lou Gehrig's disease (ALS), addictive disorders to at least one of alcohol, nicotine, and other psychoactive substance, adjustment disorder, age-associated learning and mental disorder, Anorexia nervosa, apathy, Attention-deficit disorder due to general medical conditions, Attention-deficit hyperactivity disorder, Bipolar disorder, Bulimia nervosa, Chronic fatigue syndrome, chronic or acute stress, conduct disorder, Cyclothymic disorder, dizziness, Dysthymic disorder, Fibromyalgia and other somatoform disorders, Incontinence, Inhalation disorder, Insomnia, Intoxication disorder, Obesity, Peripheral neuropathy, Premenstrual dysphoric disorder, Psychotic disorder, Seasonal affective disorder, Sexual dysfunction, Sleep disorder including narcolepsy or enuresis, Specific developmental disorder, TIC disorders including Tourette's Disease, and Withdrawal syndrome.

3. A method according to claim 1 or 2, wherein the ocular route of drug delivery is selected from the group consisting of eye-drops, suspensions, ointments, gels, hydrogels and viscosified solution systems, gel-forming systems, lotions, sprays, liposomes, emulsions, strips, therapeutic contact lenses, membrane-bound devises, collagen shields, inserts, polymeric dosing systems, rod-like inserts, iontophoresis, anterior chamber dosing, sub-conjunctival dosing or implants, sub-tenon dosing or implants, retrobulbar dosing or implants, peri-bulbar dosing or implants, trans-septal dosing or implants, choroidal dosing or implants, ciliary-body dosing or implants, sub-retinal dosing or implants, intra-vitreal dosing or implants, intraocular implantable or injected sustained release systems, encapsulated cell technology dosing systems, transscleral drug delivery systems, optic nerve related dosing systems, infusion to ocular tissue via a pump-catheter system, drug incorporation in surgical irrigating solutions and ocular dosing of gene-therapy vectors.

4. A method according to claim 1 or 2, wherein the pharmaceutical composition is a N-methyl-D-aspartate receptor antagonist.

5. A method according to claim 4, wherein the N-methyl-D-aspartate receptor antagonist is memantine.

6. A method according to claim 1 or 2, wherein the pharmaceutical composition is an alpha-2 adrenoreceptor agonist.

7. A method according to claim 6, wherein the alpha-2 adrenoreceptor agonist is brimonidine.

8. A method according to claim 6, wherein the alpha-2 adrenoreceptor agonist is an alpha-2 adrenoreceptor subtype specific agonist.

9. A method according to claim 6, wherein the alpha-2 adrenergic agonist is selected from the group consisting of imino-imidazolines, imidazolines, imidazoles, azepines, thiazines, oxazolines, guanidines, catecholamines, and derivatives thereof.

10. A method according to claim 1 or 2, wherein the pharmaceutical composition comprises a beta-blocker.

11. A method according to claim 1 or 2, wherein the pharmaceutical composition comprises established anti-cancer therapeutics, derivatives, prodrugs, codrugs, and any combinations thereof.

12. A method according to claim 1 or 2, wherein the pharmaceutical composition comprises established anti-Parkinsonian therapeutics, and combinations thereof.

13. A method according to claim 1 or 2, wherein the pharmaceutical composition comprises recombinant adeno-associated virus, other established gene-therapy vectors, other gene delivery systems, and any combinations thereof.

14. A method according to claim 1, wherein the pharmaceutical composition comprises zinc derivatives, magnesium derivatives, vitamins, or a multi-vitamins, or any combinations thereof.

15. A method according to claim 1, wherein the pharmaceutical composition comprises established ophthalmic therapeutics and their combinations, derivatives, pro-drugs, and co-drugs.

16. A method according to claim 1 or 2, wherein the pharmaceutical composition comprises one or more prostaglandine analogues, their derivatives, pro-drugs, co-drugs, and combinations thereof.

17. A method according to claim 16, wherein the prostaglandine analogue is selected from the group consisting of latanoprost, unoprostone, travaprost and bimatoprost.

18. A method according to claim 1 or 2, wherein the pharmaceutical composition comprises one or more prostamid receptor agonists.

19. A method according to claim 18, wherein the prostamid receptor agonist comprises bimatoprost.

20. A method according to claim 1 or 2, wherein the pharmaceutical composition comprises one or more one of the agonists of the cannabinoid receptors.

21. A method according to claim 1 or 2, wherein the pharmaceutical composition comprises a steroid.

22. A method according to claim 21, wherein the steroid is an angiostatic steroid.

23. A method according to claim 22, wherein the angiostatic steroid comprises Anecortave.

24. A method according to claim 1 or 2, wherein the pharmaceutical composition comprises an imino-imidazoline selected from the group consisting of clonidine and apraclonidine.

25. A method according to claim 1 or 2, wherein the pharmaceutical composition comprises an imidazoline selected from the group consisting of naphazoline, xymetazoline, tetrahydrozoline, and tramazoline.

26. A method according to claim 1 or 2, wherein the pharmaceutical composition comprises an imidazole selected from the group consisting of detomidine, medetomidine, and dexmedetomidine.

27. A method according to claim 1 or 2, wherein the pharmaceutical composition comprises an azepine selected from the group consisting of B-HT 920 (6-allyl-2-amino-5,6,7,8 tetrahydro-4H-thiazolo[4,5-d]-azepine) and B-HT 933.

28. A method according to claim 1 or 2, wherein the pharmaceutical composition comprises a thiazine.

29. A method according to claim 28, wherein the thiazine comprises xylazine.

30. A method according to claim 1 or 2, wherein the pharmaceutical composition comprises an oxazoline

31. A method according to claim 30, wherein the oxazoline is rilmenidine.

32. A method according to claim 1 or 2, wherein the pharmaceutical composition comprises a guanidine selected from the group consisting of guanabenz and guanfacine.

33. A method according to claim 1 or 2, wherein the pharmaceutical composition comprises a catecholamine.

34. A method according to claim 1 or 2, wherein the pharmaceutical composition comprises an alpha-2 adrenergic agonist comprising at least one quinoxaline component.

35. A method according to claim 34, wherein the quinoxaline components comprise quinoxaline derivatives selected from the group consisting of (2-imidozolin-2-ylamino) quinoxaline, 5-halide-6-(2-imidozolin-2-ylamino) quinoxaline, and tartrates of 5-bromo-6-(2-imidozolin-2-ylamino) quinoxaline.

36. A method according to claim 1, wherein the subject is an animal.

37. A method according to claim 1, wherein the subject is a human.

38. A method for treating migraines in humans, comprising administering to a subject in need of treatment an effective amount of a pharmaceutical composition by the ocular route of drug delivery.

39. A method according to claim 38, further comprising administering to said subject an effective amount of an established anti-migraine therapeutic agent in combination with said pharmaceutical composition.

40. A method according to claim 39, wherein the anti-migraine therapeutic agent is delivered through a systemic route.

41. A method according to claim 39, wherein the anti-migraine therapeutic agent is delivered through the ocular route of drug delivery.

42. A method according to claims 38, wherein the pharmaceutical composition comprises an alpha-2 adrenoreceptor agonist.

43. A method according to claim 40, wherein the alpha-2 adrenoreceptor agonist is an alpha-2 adrenoreceptor subtype specific agonist.

44. A method according to claim 40, wherein the alpha-2 adrenergic agonist is selected from the group consisting of imino-imidazolines, imidazolines, imidazoles, azepines, thiazines, oxazolines, guanidines, catecholamines, and derivatives thereof.

45. A method according to claim 41, wherein the alpha-2 adrenoreceptor agonist comprises brimonidine.

44. A method according to claim 41, wherein the pharmaceutical composition comprises brimonidine tartrate.

45. A method according to claim 41, wherein the brimonidine tartrate is administered through the ocular route of drug delivery in a 0.0001%-9% range of w/v composition.

46. A method for treating migraines in humans, comprising administering to a subject in need of treatment an effective amount of an established anti-migraine therapeutic agent by the ocular route of drug delivery.
